(19) United States
(12) Patent Application Publication
(10) Pub. No.: US 2006/0233737 A1
(43) Pub. Date: Oct. 19, 2006

(54) Composition comprising an extract of aphanizomenon flos-aquae, use thereof and preparation of same

(76) Inventors: Marie-Claire Janailhac, Paris (FR); Catherine Renard, Paris (FR)

Correspondence Address: BROWDY AND NEIMARK, P.L.L.C.;624 NINTH STREET, NW, SUITE 300, WASHINGTON, DC, 20001-5303, US

(21) Appl. No.: 10/542,578

(22) PCT Filed: Jan. 23, 2004
(86) PCT No.: PCT/FR04/00167

§ 371 Date: May 15, 2006

(30) Foreign Application Priority Data
Jan. 24, 2003 (FR) 03/00818

(51) Int. Cl. A61K 8/97; A61K 36/18

(52) U.S. Cl. 424/74; 424/778

(57) ABSTRACT

The invention relates to a topical composition comprising at least one extract of Aphanizomenon flos-aquae flos-aquae at a concentration of between 0.01 and 10% dry matter in relation to the total weight of the composition. The inventive composition is used to treat the upper layers of the epidermis and/or the hair.

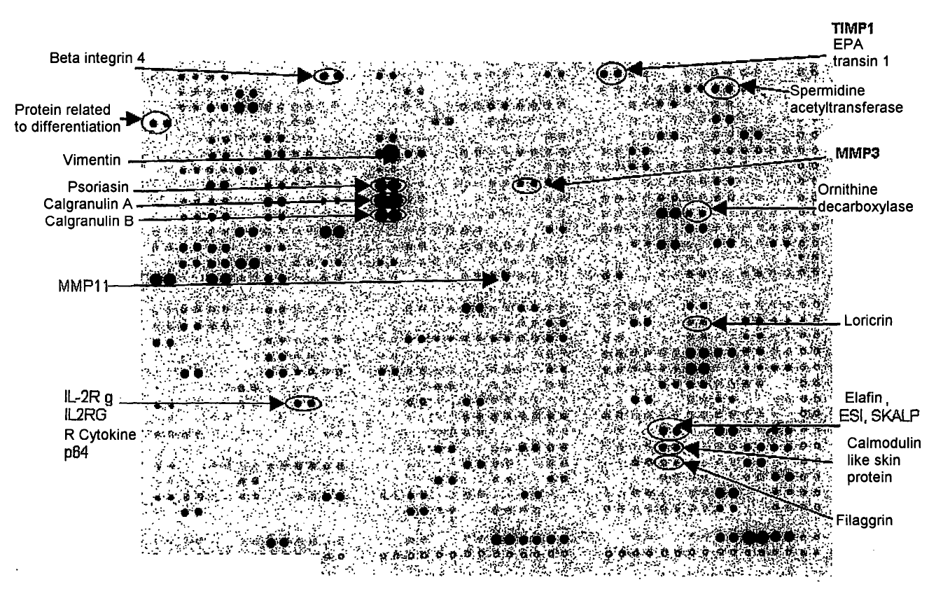

The present invention relates to a composition comprising an extract of the aphanizomenon-flos-aquae flos-aquae alga, which may be applied topically. It applies more particularly but not exclusively, to the treatment of the upper layers of the epidermis and/or of hair, notably for preventing and treating induced skin ageing and/or for repairing certain changes in skin tissues such as stretch marks and/or for contributing to improving the hair's aspect.

Generally, induced skin ageing is caused by extrinsic factors (photo-induced and environmental ageing). At the level of the skin, exposure to the environment whether it be to the sun or to atmospheric pollutants finds its expression in deep wrinkles and wrinklets, telangiectasies and purpuric lesions, pigmentary spots and sebaceous hyperplasia, skin hydration disorders, increase in transepidermic water loss, changes in surface lipids, increase in skin desquamation.

The most important histological changes related to induced ageing are located at the dermis, notably at the fibroblasts, components of the extracellular matrix and vascular network. The dermo-epidermic junction collapses causing a reduction in the succession points between the dermis and the epidermis. A simultaneous change in the cells of the epidermis is also noted with a polarity loss of keratinocytes, a reduction of the number of Langherans cells, etc. . . .

The present invention relates to the use of a unique variety of cyanobacteria, a variety discovered in Lake Klamath, Oreg. (USA) and characterized by Renhui et al. (Renhui Li, Wayne W. Carmichael, Yongding Lui & Makoto M. Watanabe, Hydrobiology, 438: pages 99-105, 2000, Taxonomic reevaluation of Aphanizomenon-flos-aquae NH-5 based on morphology and 16S rRNA gene sequences).

Cyanobacteria form a particular group of prokaryotes formerly related to the vegetable kingdom. These are very small often blue-green (hence the name of blue algae) unicellular living cells, autotrophic and with a relatively simple organization. They have the capacity of growing in extreme media and fixing dinitrogen.

Preparations based on dried Aphanizomenon-flos-aquae flos-aquae are recommended as a food complement for their numerous constituents, notably their high content in highly assimilable proteins and the presence of vitamins B6, B 12 and F.

Indeed, the listed investigations show that oral administration of Aphanizomenon-flos-aquae flos-aquae:
allows an increase in the reactivity of the immune system by increasing the synthesis of messenger RNA coding for interleukine 1 (IL-1) (Characterization of human monocyte activation by a water soluble preparation of Aphanizomenon-flos-aquae, Phytomedicine, Pugh N, Pasco DS, 2001, Nov. 8(6): pages 445-53),
is beneficial to health by the diversity of the nutrients which compose it (Microalgae as food & supplement, Kay R A, Crit. Rev. Food Sci. Nut., 1991, 30(6): pages 555-73),
is a good nutritional source of polyunsaturated fatty acids which give it a hypocholesterolemic property (Rafial I. Kushak, Christian Drapeau, Elisabeth M. Van Cott, Harland H; Winter, JANA, vol. 2(3), 2000, pages 59-65).

On the other hand, no document refers to the use of Aphanizomenon-flos-aquae flos-aquae in the prerparation of beneficial compositions for preventing skin ageing and improving hair aspect, notably for a topical application.

Now, a per se incorporation of Aphanizomenon-flos-aquae flos-aquae in topically usable compositions is not possible considering the low level of solubilization of the dried alga, its strong coloration, its strong smell and the lack of stability of its biochemical compounds.

Therefore, the object of the invention is to solve these drawbacks by developing a topically applicable composition which allows the active ingredients of Aphanizomenon-flos-aquae flos-aquae to be retained in all their integrity so as to be actively involved in treating the upper layers of the epidermis and/or of hair, notably for preventing skin ageing and improving hair aspect.

For this purpose, it proposes a topically applicable composition comprising at least one extract of Aphanizomenon-flos-aquae flos-aquae at a concentration between 0.01 and 10% by dry weight relatively to the total weight of the composition.

Advantageously, a method for preparing said composition comprising at least one extract of Aphanizomenon-flos-aquae flos-aquae comprises the preparation of said extract by extracting active substances contained in for example dry, dried freeze-dried Aphanizomenon-flos-aquae flos-aquae notably according to the following steps:
at least one maceration at a temperature from 25 to 50° C. of dried blue algae of the Aphanizomenon-flos-aquae flos-aquae species in the presence of enzymes such as cellulases, pectinases and glucanases for a time from ten minutes to ten hours under stirring,
a liquid/solid separation by centrifugation,
a liquid/liquid separation by a membrane filtration method,
drying and/or dilution in a solution containing specific adjuvants, for example sorbitol,
an optional specific separation of the different thereby extracted constituents for example by chromatography, the different obtained substances able to be used either alone or as a mixture, according to the sought-after effect.

The drying step may both be a standard (heat) drying step and a drying step by nebulization or freeze-drying.

With this method, the substances having a major activity on the skin and hair are extracted, i.e.:
carotenoids,
phycocyanins,
amino acids, notably methionine, lysine, proline and serine,
polysaccharides.

The aforesaid extract may be dissolved in an aqueous solution such as a water/sorbitol mixture.

Said composition may exist as simple or multiple emulsions such as a water/oil or oil/water emulsion, or even a triphasic emulsion and/or a gel or an aqueous or hydro-alcoholic solution.

Said composition may also exist as a vectorized system with controlled release or modulated release.

Embodiments of the invention and exemplary formulations of said dermatologic cosmetic product and/or composition will be described hereafter as non-limiting examples.

The unique FIGURE is the illustration of a diagram of a profile obtained by hybridization of complementary DNA probes, marked with different mRNAs obtained with a normal human epidermis treated with a raw aqueous extract of Aphanizomenon-flos-aquae flos-aquae.

The method for preparing a composition comprising at least one extract of Aphanizomenon-flos-aquae flos-aquae containing active substances comprises the preparation of said extract according to the following steps:
at least one maceration at a temperature from 25 to 50° C. and preferably 35° C., of dried blue Aphanizomenon-flos-aquae flos-aquae algae in the presence of cellulases, pectinases, and glucanases for a time from ten minutes to ten hours, and preferably four hours under stirring. The results of the tests show that the attack by the different enzymes provides better solubilization of the parenchymatic wall of the algae and thus a higher polysaccharide richness of the thereby prepared aqueous extract.
a liquid/solid separation by centrifugation under an acceleration from 5,000 to 10,000 g, and preferably 9,000 g.
a liquid/liquid separation by a membrane filtration method with a cutoff threshold between 100,000 Daltons and 0.2 μm.
drying and/or dilution in an aqueous solution of sorbitol. By drying, is meant both standard drying (heat) and drying by nebulization or freeze-drying.
Specific separation of the different constituents, thereby extracted by chromatography, the different obtained substances being used alone or as a mixture according to the sought-after effect.

The thereby obtained extract notably consists of proteins (between 0.05 to 1% m/m (mass ratio)), vitamin B12 (between 0.003 to 0.05% m/m), lysine (between 0.2 to 3% m/m), methionine (between 0.04 to 0.6% m/m), proline (between 0.15 to 2.5% m/m) and serine (between 0.15 to 2.5 m/m).

The high density filter or cDNA macroarray method on a support comprising at least 600 characteristic genes of the skin and pilous system was used for investigating the effect of the Aphanizomenon-flos-aquae flos-aquae extract on the expression of genes coding for major proteins of cosmetic or dermo-cosmetic interest.

In this method, the deposits (probes) are cDNA (complementary deoxyribonucleic acid) clones or PCR (Polymerase Chain Reaction) products, fixed at a high density on a nylon membrane. Marking is most frequently radioactive and screening is achieved with an excess of the target, a measurement of the relative abundance of each of the mRNAs (messenger ribonucleic acid) present in the starting sample is thereby obtained.

The proteins are obtained from skin explants prepared following a mammary plasty on a donor.

Two skin samples of 20 cm² were prepared and maintained alive in a culture medium.

The Aphanizomenon-flos-aquae flos-aquae extract was applied on the explants in an amount of 5 mg/cm² of a 2% raw aqueous extract solution without any adjuvant, mornings and evenings for two days.

After each application, the explants were incubated at 37° C. with 5% of CO₂.

The control skin explant was treated according to the same method with sterile water.

The skin pieces (epidermises) were rinsed and then placed in the presence of Tri-reagent® (Sigma T9424) and then frozen at −80° C.

The RNAs were extracted and purified from the supernatants obtained after milling the frozen epidermises.

The RNAs are treated by means of a RNAse-out enzyme on the one hand in order to inhibit the RNAse enzymes, and, on the other hand, by means of a DNAse 1 enzyme for eliminating traces of DNA contaminating the RNA.

The quality of the RNAs is checked by electrophoresis on agarose gel.

The mRNA groups are purified by hybridization of the poly(A) ends (chain of adenosine nucleotides) of the mRNAs with biotinylated oligo(dT) (oligonucleotides consisting of deoxythymidines) primers.

Multiple DNA probes marked with phosphorus ³³P were produced by inverse transcription of mRNAs bound on poly(dT) (chain of deoxythymidine nucleotides) beads, by a group of specific primers of immobilized sequences on the filters in presence of α³³P dATP (α³³P-deoxyadenosine triphosphate).

The marked probes were purified by exclusion chromatography also called molecular sieving, gel-filtration or gel permeation chromatography.

The quality and the equivalence of the marked probes were evaluated by liquid scintillation counting. Scintillators are media in which a not insignificant fraction of the absorbed energy during an interaction with an α or β particle is converted by luminescence into photons able to be detected. This detection consists in converting them into an electrical signal which may be processed by suitable electronics.

Membranes of the Custom ATLAS BA 600/1 type are pretreated and then the cDNAs immobilized on each membrane are hybridized (68° C., 12 hours) with corresponding marked probes, the filters are then washed and analyzed by direct quantification of the radioactivity of the spots by means of a phosphorimager® (Cyclone, Packard Instrument) type of apparatus and its QuantArray® (Packard) software.

Table I below shows the genes, the relative expression (RE) of which was significantly changed after forty-eight hours of a bidaily application of a raw extract of Aphanizomenon-flos-aquae flos-aquae on a normal human epidermis.

TABLE I

| | | Aphanizomenon-flos-aquae | |
|---|---|---|---|
| | Control | flos-aquae extract | |
| Name of the genes | RE | RE | % |
| Vimentin (VIM) | 10.0 | 21.8 | 217 |
| Metalloprotease 11 (MMP11) | 6.2 | 15.9 | 255 |
| Stromelysine 3 | | | |
| Metalloprotease 3 (MMP3); | 5.7 | 17.3 | 306 |
| Stromelysine 1 (STMY1; SL1); | | | |
| Transin 1 | | | |
| Tissular inhibitor of | 10.0 | 24.3 | 244 |
| metalloprotease 1 (TIMP1); | | | |
| Erythroid potentiator | | | |
| activity (EPA); Inhibitor of | | | |
| fibroblastic collagenases | | | |
| Gamma sub-unit of the | 6.6 | 20.0 | 302 |
| interleukine-2 receptor | | | |
| (IL-2R gamma; IL2RG); Common | | | |
| receptor of gamma chains | | | |
| of cytokines; P64 | | | |
| Epidermal filaggrin (FLG) | 28.7 | 7.3 | 25 |
| Loricrin (LOR; LRN) | 31.9 | 11.7 | 37 |
| Protein related to | 15.8 | 23.1 | 146 |
| differentiation of | | | |
| adipocytes | | | |
| Beta integrin (ITGB4); | 24.7 | 40.1 | 162 |
| antigen CD104 | | | |
| S100 A7 protein binding | 135.2 | 202.0 | 149 |
| calcium; psoriasin | | | |
| S100 A8 protein binding | 311.1 | 485.4 | 156 |
| calcium (S100A8); | | | |
| Calgranulin A (CALA); | | | |
| Migration inhibitory | | | |
| factor-related protein | | | |
| 8 (MRP8); Leukocyte L1 | | | |
| complex light chain; | | | |
| Cystic fibrosis antigen | | | |
| (CFAG) | | | |
| S100 A9 protein binding | 210.2 | 323.8 | 154 |
| calcium (S100A9); | | | |
| Calgranulin B (CAGB); | | | |
| Migration inhibitory | | | |
| factor-related protein | | | |
| 14 (MRP 14); Leukocyte | | | |
| L1 complex light chain; | | | |
| Ornithine decarboxylase | 9.4 | 18.1 | 193 |
| (ODC) | | | |
| Spermidine | 13.9 | 26.3 | 189 |
| acetyltransferase | | | |
| Elafin; specific inhibitor | | | |
| of elastases (ESI); | | | |
| skin-derived | 22.6 | 34.3 | 152 |
| antileukoproteinase | | | |
| (SKLP) | | | |
| Calmodulin-like skin | 31.8 | 16.0 | 50 |
| protein (CLSP) | | | |

The diagram of the unique FIGURE illustrates the profile obtained by hybridization of complementary DNA probes marked with different mRNAs obtained with normal human epidermis treated with a raw aqueous extract of Aphanizomenon-flos-aquae flos-aquae.

The expression of the genes shown in bold script in the diagram of the unique FIGURE (calgranulin A, calgranulin B, loricrine, psoriasin, a protein of the calmodulin type, MMP3 and TIMP1) was also quantified by the RT-PCR (Reverse Transcriptase Polymerase Chain Reaction) technique in order to validate the first obtained results.

In this experiment, actin was used as a standard marker.

The results shown in Table II below were obtained:

| | Expression of the gene |
|---|---|
| | relatively to the control |
| Gene | (base 100)-RT-PCR |
| Calgranulin A | 153 |
| Calgranulin B | 166 |
| Filaggrin | 16 |
| Loricrin | 14 |
| Psoriasin | 137 |
| Calmodulin-like Skin Protein | 44 |
| Metalloprotease 3 matrix | 230 |
| Tissular inhibitor of metalloprotease 1 | 209 |

The treatment of skin explants by an extract of Aphanizomenon-flos-aquae flos-aquae induces significant changes in the expression of the differentiation and proliferation of cells of the epidermis. These changes are identical with those obtained with a compound of the retinol (or retinoid: lipid which directly diffuses into the plasmic membrane) type without the Aphanizomenon-flos-aquae flos-aquae extract having the formulation constraints.

Expression of CLSP (Calmodulin-Like Skin Protein), a specific marker of the differentiation of keratinocytes, is considerably repressed by retinoids and their analogs in the stratum granulosum (the third most internal layer of the epidermis where keratin appears as granules) and in the lower layers of stratum corneum (the most external layer of the epidermis) (Mehul B. et al., Calmodulin-like skin protein: a new marker of keratinocyte differentiation, J. Invest. Dermatol., 2001 Jun., 116(6), 905-9).

The raw extract of Aphanizomenon-flos-aquae flos-aquae reduces the expression of CLSP, twice; this is an argument in favor of its involvement in the modulation of this marker.

Moreover retinoids inhibit the expression of loricrin (Brown L. J. et al., Retinoic acid suppression of loricrin expression in reconstituted human skin cultured at the liquid-air interface, J. Invest. Dermatol., 1994 June, 102(6), 886-90), as well as the raw extract of Aphanizomenon-flos-aquae flos-aquae which reduces its expression by more than 10 fold.

As for filaggrins, which further result from the digestion of the proteins of profilaggrins contained in the granules in the lower portion of stratum corneum, they are then digested by peptidases into amino-acids. The inhibition of the messengers of filaggrins may result from a global inhibition of keratinocyte differentiation and thereby contribute to better cohesion of the corneocytes (in the corneal layer, the keratinocyte is named corneocyte) and therefore to an improvement of the skin barrier function.

Loricrin is the major constituent of the wall of corneocytes, and is contained in the granules up to the terminal stage of the differentiation and then contributes to the formation of the envelope of the corneocytes in order to strengthen it. Reduction of its expression under the effect of the raw extract of Aphanizomenon-flos-aquae flos-aquae is consistent with the development of expressions of filaggrins and CLSP.

On the other hand, expression of calgranulins A and B, which are synthesized by the epithelial cells and keratinocytes, is increased under the effect of the raw extract of Aphanizomenon-flos-aquae flos-aquae. Psoriasin which like calgranulin A and calgranulin B, belongs to the S100 protein family, and the expression of which is inducible by retinoids (Tavakkov A. et al., a retinoic acid-inducible skin-specific gene (TIS-1/psoriasin): molecular cloning and analysis of gene expression in human skin in vivo and cultured skin cells in vitro, Mol. Biol. Rep., 1994, 20(2), 75-83) in differentiating primary keratinocytes, has an expression which also increases under the effect of the treatment. The same applies to the increase of the expression of MMP3 which is known to be significantly increased under the effect of retinoids (Varani J. et al., Expression of serine proteases and metalloproteinases in organ-cultured human skin. Altered levels in the presence of retinoic acid and possible relationship to retinoid-induced loss of epidermal cohesion, Am. J. Pathol., 1994, 145, 561-573.

All these events—activation of the relative expression of the messengers calgranulin A, calgranulin B, psoriasin, metalloprotease 3 and inhibition of the expression of messengers filaggrin, loricrin, calmodulin-like skin protein—let us anticipate a retinoid-like action of the topical application of the raw extract of Aphanizomenon-flos-aquae flos-aquae. Further, the increase in the expression of the tissular inhibitor of metalloprotease 1 (TIMP1) assumes an anti-ageing effect during topical application of a cosmetic composition based on Aphanizomenon-flos-aquae flos-aquae.

A composition for after-sun care comprises:

| | | |
|---|---|---|
| A1* | Demineralized water | qs**100% |
| A2 | Sequestrene ®NA4/Celon ®E/Trilon ®B | 0.01% |
| B1 | Nipagin ®M/Methyl-POB | 0.05% |
| C1 | Carbopol ®940 | 15.00% |
| D1 | Triethanolamine | 0.5-1% |
| E1 | Antimicrobial preservative | 0.5-1% |
| F1 | Silicone | 1-2% |
| F2 | Perfume | 0.15% |
| G1 | Aphanizomenon-flos-aquae flos-aquae extract | 0.5-5% |
| | In solution in sorbitol and water (i.e. 1-5% | |
| | of dry extract of Aphanizomenon-flos-aquae | |
| | flos-aquae) | |

(*each of the letters placed in front of a component represents a phase)
(**qs: quantum satis)

A composition for anti-ageing care comprises:

| | | |
|---|---|---|
| A1* | Emulium ® (Gattefossé) | 4.0% |
| A2 | Amercol ® (Amerchol) | 6.0% |
| A3 | Amerlate ® (Amerchol | 2.0% |
| A4 | Oily calendula Végétol ® (Gattefossé) | 2.0% |
| A5 | LNST ®98 (Lanatech) | 1.0% |
| B1 | Demineralized water | qs 100% |
| B2 | Carbopol ® (B F Goodrich) | 10.0% |
| C | Abil ® (Goldschmidt) | 6.0% |
| D1 | Demineralized water | 5.0% |
| D2 | Triethanolamine (Prolabo) | 0.2% |
| E1 | Antimicrobial preservative | 0.5% |
| E2 | Natural glycerin (Elf Atochem) | 4.0% |
| F | Fluidamid ®DF125 (Roquette) | 4.0% |
| G | Aphanizomenon-flos-aquae flos-aquae extract | 0.5-5% |
| | in solution in sorbitol and water (i.e. | |
| | 1-5% of dry Aphanizomenon-flos-aquae | |
| | flos-aquae extract) | |
| H | Perfume | 0.3% |

A composition for washing and taking care of hair comprises:

| | | |
|---|---|---|
| A1* | Texapon ® (Henkel) | 10.00% |
| B1 | Demineralized water | qs 100% |
| B2 | Sequestrene ® (Prolabo) | 0.05% |
| C1 | Tegobetain ® (Goldschmidt) | 10.00% |
| D1 | Emilan ® (Albright & Wilson St Mihiel) | 4.00% |
| E1 | Hydralphatin ®3P (Lanatech) | 3.00% |
| E2 | Antimicrobial preservative | 0.50% |
| F1 | Glutamate (Amerchol) | 15.00% |
| G1 | Oramix ® (Seppic) | 6.00% |
| G2 | Simulson ® (Seppic) | 1.00% |
| H1 | Demineralized water | 5.00% |
| H2 | Acrylsol ® (Seppic) | 6.00% |
| J1 | Aphanizomenon-flos-aquae flos-aquae extract | 0.5-5% |
| | in solution in sorbitol and water (i.e. | |
| | 1-5% of dry Aphanizomenon-flos-aquae | |
| | flos-aquae extract) | |

An antiwrinkle care composition comprises:

| | | |
|---|---|---|
| A1* | Demineralized water | qs**100% |
| A2 | Sepigel ® (Seppic) | 1.0% |
| B1 | Emulium ® (Gattefossé) | 3.0% |
| B2 | Amerchol ® (Amerchol) | 4.0% |
| B3 | Crodamol ® (Croda) | 8.0% |
| B4 | Abil ® (Goldschmidt) | 5.0% |
| C | Antimicrobial preservative | 0.3% |
| D | Fluidamid ® DF15 (Gattefossé) | 3.0% |
| E | Aphanizomenon-flos-aquae flos-aquae extract | 0.5-5% |
| | in solution in sorbitol and water (i.e. | |
| | 1-5% of dry Aphanizomenon-flos-aquae | |
| | flos-aquae extract) | |

A treatment mask composition for dried hair comprises:

| | | |
|---|---|---|
| A1 | Cetearyl Glucoside (Montanov ® 68-SEPPIC) | 7% |
| A2 | Coco betaine (AMONYL ® 265BA-SEPPIC) | 0.5% |
| A3 | Shea butter | 4% |
| A4 | Beeswax | 2% |
| A5 | Dimethicone (DOW CORNING) | 5% |
| B1 | Demineralized water | qs 100% |
| B2 | Decyl glucoside (ORAMIX ® NS10-SEPPIC) | 1% |
| C1 | Perfume | 0.5% |
| C2 | Antimicrobial preservative | 0.5% |
| C3 | Aphanizomenon-flos-aquae flos-aquae extract | 0.5-5% |
| | in solution in sorbitol and water (i.e. | |
| | 1-5% of dry Aphanizomenon-flos-aquae | |
| | flos-aquae extract) | |

A night cream comprises:

| | | |
|---|---|---|
| A1* | Cetearyl glucoside (Montanov ® 68-SEPPIC) | 6% |
| A2 | Vegetable oils | 20% |
| A3 | DL-alpha-tocopherol (BASF) | 0.05% |
| B1 | Demineralized water | qs 100% |
| C1 | Antibacterial preservative | 0.5% |
| C2 | Perfume | 0.3% |
| C4 | Aphanizomenon-flos-aquae flos-aquae extract | 0.5-5% |
| | in solution in sorbitol and water (i.e. | |
| | 1-5% of dry Aphanizomenon-flos-aquae | |
| | flos-aquae extract) | |

1. A topically applicable composition,
characterized in that it comprises at least one Aphanizomenon-flos-aquae flos-aquae extract at a concentration between 0.01 and 10% by dry weight of the total weight of the composition.

2. The composition according to claim 1,
characterized in that it exists in the form of simple or multiple emulsions such as a water/oil emulsion or an oil/water emulsion or even a triphasic emulsion, and/or a gel, or an aqueous or hydroalcoholic solution.

3. The composition according to claim 1,
characterized in that it exists in the form of a vectorized system with controlled release or modulated release.

4. The use of a composition according to claim 1, in order to make products for treating upper layers of the epidermis and/or hair.

5. The use of a composition according to any of claims 1 to 4, for making a cosmetic.

6. The use of a composition according to any of claims 1 to 4, for making a dermatological composition.

7. A composition for after-sun care, characterized in that it comprises:

| | |
|---|---|
| Demineralized water | qs*100% |
| Sequestrene ®NA4/Celon ®E/Trilon ®B | 0.01% |
| Nipagin ®M/methyl-POB | 0.05% |
| Carbopol ®940 | 15.00% |
| Triethanolamine | 0.5-1% |
| Antimicrobial preservative | 0.5-1% |
| Silicone | 1-2% |
| Perfume | 0.15% |
| Aphanizomenon-flos-aquae flos-aquae extract | 0.5-5% |
| in solution in sorbitol and water (i.e. | |
| 1-5% of dry Aphanizomenon-flos-aquae | |
| flos-aquae extract) | |

8. A composition for after-sun care, characterized in that it comprises:

| | |
|---|---|
| Emulium ® | 4.0% |
| Amerchol ® | 6.0% |
| Amerlate ® | 2.0% |
| Oily calendula Vegetol ® | 2.0% |
| LNST ® 98 | 1.0% |
| Demineralized water | qs 100% |
| Carbopol ® | 10.0% |
| Abil ® | 6.0% |
| Demineralized water | 5.0% |
| Triethanolamine | 0.2% |
| Antimicrobial preservative | 0.5% |
| Natural glycerin | 4.0% |
| Fluidamid ®DF125 | 4.0% |
| Aphanizomenon-flos-aquae flos-aquae extract | 0.5-5% |
| in solution in sorbitol and water (i.e. | |
| 1-5% of dry Aphanizomenon-flos-aquae | |
| flos-aquae extract) | |
| Perfume | 0.3% |

9. A composition for washing and taking care of hair,
characterized in that it comprises:

| | |
|---|---|
| Texapon ® | 10.00% |
| Demineralized water | qs 100% |
| Sequestrene ® | 0.05% |
| Tego betain ® | 10.00% |
| Empilan ® | 4.00% |
| Hydralphatine 3P | 3.00% |
| Antimicrobial preservative | 0.50% |
| Glutamate | 15.00% |
| Oramix ® | 6.00% |
| Simulsol ® | 1.00% |
| Demineralized water | 5.00% |
| Acrysol ® | 6.00% |
| Aphanizomenon-flos-aquae flos-aquae | 0.5-5% |
| extract in solution in sorbitol and water | |
| (i.e. 1-5% of dry Aphanizomenon-flos-aquae | |
| flos-aquae extract) | |

10. An antiwrinkle composition,
characterized in that it comprises:

| | |
|---|---|
| Demineralized water | qs 100% |
| Sepigel ® | 1.0% |
| Emulium ® | 3.0% |
| Amerchol ® | 4.0% |
| Crodamol ® | 8.0% |
| Abil ® | 5.0% |
| Antimicrobial preservative | 0.3% |
| Fluidamid ®DF15 | 3.0% |
| Aphanizomenon-flos-aquae flos-aquae | 0.5-5% |
| extract in solution in sorbitol and water | |
| (i.e. 1-5% of dry Aphanizomenon-flos-aquae | |
| flos-aquae extract) | |

11. A treatment mask composition for dried hair characterized in that it comprises:

| | |
|---|---|
| Cetearyl glucoside | 7% |
| Coco betaine | 0.5% |
| Shea butter | 4% |
| Beeswax | 2% |
| Dimethicone | 5% |
| Demineralized water | qs 100% |
| Decyl glucoside | 1% |
| Perfume | 0.5% |
| Antimicrobial preservative | 0.5% |
| Aphanizomenon-flos-aquae flos-aquae | 0.5-5% |
| extract in solution in sorbitol and water | |
| (i.e. 1-5% of dry Aphanizomenon-flos-aquae | |
| flos-aquae extract) | |

12. A night cream,
characterized in that it comprises:

| | |
|---|---|
| Cetearyl glucoside | 6% |
| Vegetable oils | 20% |
| DL-alpha tocopherol | 0.05% |
| Dimineralized water | qs 100% |
| Antimicrobial preservative | 0.5% |
| Perfume | 0.3% |
| Aphanizomenon-flos-aquae flos-aquae | 0.5-5% |
| extract in solution in sorbitol and water | |
| (i.e. 1-5% of dry Aphanizomenon-flos-aquae | |
| flos-aquae extract) | |

13. A method for preparing a composition according to claim 1, characterized in that it comprises the following steps:
at least one maceration at a temperature from 25 to 50° C. and, preferably 35° C., of dried Aphanizomenon-flos-aquae flos-aquae blue algae in the presence of enzymes such as cellulases, pectinases, and glucanases, for a time from ten minutes to ten hours, and preferably for four hours under stirring,
a liquid/solid separation by centrifugation,
a liquid/liquid separation by a membrane filtration method,
drying and/or dilution in a solution containing specific adjuvants for example sorbitol,
optional specific separation of the different thereby extracted constituents, for example by chromatography, the different substances obtained may be used alone or as a mixture.

14. The method according to claim 13,
characterized in that the drying step is a standard (heat) drying step or a drying step by nebulization or freeze-drying.

15. The method according to claim 13,
characterized in that the aforesaid extract is dissolved in an aqueous solution such as a water/sorbitol mixture.
